(19) United States
(12) Patent Application Publication
(10) Pub. No.: US 2026/0034364 A1
(43) Pub. Date: Feb. 5, 2026

(54) INTRA-LUMINAL MEDICAL DEVICE WITH EVOKED BIOPOTENTIAL SENSING CAPABILITY

(71) Applicant: MEDTRONIC, INC., Minneapolis, MN (US)

(72) Inventors: Varun Umesh Kashyap, Palo Alto, CA (US); David A. Dinsmoor, North Oaks, MN (US); Kristin N. Hageman, Dayton, MN (US); Scott R. Stanslaski, Shoreview, MN (US); Mark Ashby, Laguna Niguel, CA (US)

(21) Appl. No.: 19/099,538

(22) PCT Filed: Jul. 18, 2023
(86) PCT No.: PCT/IB2023/057333

§ 371 Date: Jan. 29, 2025

Related U.S. Application Data
(60) Provisional application No. 63/369,810, filed on Jul. 29, 2022.

(51) Int. Cl. A61N 1/36

(52) U.S. Cl. CPC A61N 1/36139

(57) ABSTRACT

Sensing an evoked response to electrical stimulation of target tissue of a patient in conjunction with an intra-luminal electrode. The intra-luminal electrode may be implanted in a blood vessel or similar lumen proximal to the target tissue and the sensed signals and/or delivered stimulation may pass through the blood vessel. or other lumen, walls. In some examples the evoked response may be an evoked compound action potential (ECAP), which may also be evoked resonant neural activity (ERNA). The electrical stimulation may elicit a measurable response indicative of a thought pattern or neural state that would otherwise be undetectable using a non-evoked biopotential.

This application claims the benefit of U.S. Provisional Patent Application Ser. No. 63/369,810, filed 29 Jul. 2022, the entire content of which is incorporated herein by reference.

### TECHNICAL FIELD

The disclosure relates to sensing and stimulation within a lumen of anatomical structure.

### BACKGROUND

Medical devices may be external or implanted and may be used to sense bioelectrical signals and to deliver electrical stimulation therapy to various tissue sites of a patient to treat a variety of symptoms or conditions such as chronic pain, tremor, Parkinson's disease, other movement disorders, epilepsy, urinary or fecal incontinence, sexual dysfunction, obesity, or gastroparesis. A medical device delivers may sense electrical activity, such as local field potential (LFP) activity of a patient as well as deliver electrical stimulation therapy via one or more leads that include electrodes located proximate to target locations associated with the brain, the spinal cord, pelvic nerves, peripheral nerves, or the gastrointestinal tract of a patient. For bipolar stimulation, the electrodes used for stimulation may be on one or more leads. For unipolar stimulation, the electrodes may be on one or more leads, and an electrode on a pulse generator housing located remotely from the target site (e.g., near clavicle). It may be possible to use leadless stimulation using electrodes mounted on the pulse generator housing. Electrical stimulation and sensing may be used in different therapeutic applications, such as deep brain stimulation (DBS), spinal cord stimulation (SCS), pelvic stimulation, gastric stimulation, or peripheral nerve field stimulation (PNFS).

### SUMMARY

In general, the disclosure describes techniques for sensing an evoked biosignal in conjunction with an intra-luminal electrode system. In some examples, the evoked biosignal may include an evoked compound action potential (ECAP), an evoked compound muscle action potential (ECMAP), or evoked resonant neural activity (ERNA). The evoked compound action potential (ECAP), for example, is the synchronized activation of excitable tissue elicited with at least a single stimulus, although multiple stimuli may be delivered to potentiate effects in the neural substrate. An intra-luminal electrode of this disclosure is an electrode that may be implanted in a lumen of a blood vessel or within any other lumen of an anatomical structure near target tissue, including nerve tissue. The electrode may connect to sensing and/or electrical stimulation circuitry of a medical device. The sensed signals and/or delivered stimulation may pass through the wall of a blood vessel, or other anatomical structure.

In some examples, biopotential signal amplitudes may be attenuated, and stimulation coupling into the target tissue may be reduced, because of the insulating effect of the body lumen walls. A medical system of this disclosure may sense a biopotential such as an ERNA with the intra-luminal electrode to, for example, detect a measurable response indicative of a thought pattern or neural state that would otherwise be undetectable using a non-evoked biopotential, such as a local field potential. In other examples, the medical system may elicit biopotentials such as ECAPS, ERNAs and ECMAPs as an aid for determining the optimal location for sensing biopotentials that are indicative of thought patterns or neural states. The medical system may also be configured to deliver stimulation priming signals that enhance the detectability of relevant thought patterns or neural states and assess the patency of the intra-luminal medical device. The patency of the intra-luminal medical device may include its operational function, in a known location, and an intimate contact with the anatomical targets of interest.

In one example, this disclosure describes a system comprising: an electrode configured to be located within a lumen of an anatomical structure and proximal to target tissue of a patient; a medical device comprising: electrical stimulation circuitry configured to deliver electrical stimulation to the patient; sensing circuitry configured to detect bioelectrical signals from the patient via the electrode; and processing circuitry configured to: control the electrical stimulation circuitry to deliver electrical stimulation to the patient; receive, via the sensing circuitry, an indication of a biopotential that was elicited by the delivered electrical stimulation, wherein the sensed biopotential is a measurable response indicative of at least one of: a thought pattern from the patient; or a neural state of the patient at the time the electrical stimulation was delivered.

In another example, this disclosure describes a method comprising: delivering electrical stimulation to target tissue of a patient; receiving, by sensing circuitry of a medical device, bioelectrical signals from the patient via one or more electrodes proximal to the target tissue, wherein at least one electrode is configured to be located within a lumen of an anatomical structure and proximal to the target tissue; receiving, by processing circuitry of the medical device, and via the sensing circuitry an indication of a biopotential that was elicited by the delivered electrical stimulation, wherein the sensed biopotential is a measurable response indicative of at least one of: a thought pattern from the patient; or a neural state of the patient at the time the electrical stimulation was delivered.

In another example, this disclosure describes a medical device comprising: electrical stimulation circuitry configured to deliver electrical stimulation to a patient via one or more electrodes, wherein at least one electrode is configured to be located within a lumen of an anatomical structure and proximal to target tissue of the patient; sensing circuitry configured to detect bioelectrical signals from the patient via the electrode configured to be located intra-luminally; and processing circuitry configured to: control the electrical stimulation circuitry to deliver electrical stimulation to the patient; receive via the sensing circuitry an indication of a biopotential that was elicited by the delivered electrical stimulation, wherein the sensed biopotential is a measurable response indicative at least one of: a thought pattern from the patient; or a neural state of the patient at the time the electrical stimulation was delivered.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example system that includes an implantable medical device (IMD) configured to deliver electrical stimulation to a patient and sense bioelectrical signals from the patient according to an example of the techniques of the disclosure.

FIG. 2A is a conceptual diagram illustrating an example electrode configured to be located intra-luminally and proximal to target tissue of a patient, according to one or more techniques of this disclosure.

FIG. 2B is a conceptual diagram of an example device including one or more electrode configured to be located within a lumen of an anatomical structure and proximal to target tissue of a patient.

FIGS. 2C and 2D are a conceptual diagrams of an example device including one or more electrode configured to spiral along a lumen of an anatomical structure and proximal to target tissue of a patient.

FIG. 3 is a block diagram of the example IMD of FIG. 1 for delivering electrical stimulation and receiving bioelectrical signals from target tissue of a patient.

FIG. 4 is a block diagram of the external programmer of FIG. 1 for controlling sensing of bioelectrical signals and the delivery of electrical stimulation according to an example of the techniques of the disclosure.

FIG. 5A is a time graph of example evoked compound action potentials (ECAPs) sensed for respective stimulation pulses, in accordance with one or more techniques of this disclosure.

FIG. 5B is a time graph illustrating an example evoked resonant neural activity (ERNA) received by an intra-luminal medical device according to an example of the techniques of the disclosure.

FIG. 6A is a time graph of example evoked compound action potentials (ECAPs) sensed for respective stimulation pulses, in accordance with one or more techniques of this disclosure.

FIG. 6B is a time graph illustrating an example of resonant bioelectrical signals evoked from electrical stimulation, according to one or more techniques of this disclosure.

FIG. 7 is a flow chart illustrating one example operation of the system of this disclosure.

### DETAILED DESCRIPTION

The disclosure describes a medical system and techniques for sensing evoked biopotentials such as ECAPs, ECMAPS and ERNAs with an intra-luminal electrode device as part of the sensing system. In some examples, the intra-luminal electrode device may include one or more electrodes. In some examples, the medical system may include one or more intra-luminal electrode devices implanted in a patient as well as other implanted electrodes.

As noted above, a sensed ECAP represents a synchronized response generated by a group of electrically activated nerve fibers. The ECAP is a signal recorded from a nerve trunk made up of numerous axons. It is the result of summation of many action potentials from the individual axons in the nerve trunk. An ECAP may be initiated on a peripheral nerve by an electrical stimulus applied to the nerve at some point at a distance from the recording site. The latency between the application of the stimulus and the onset of the compound action potential is a function of the distance between the recording site and the site of stimulation. The amplitude of the recorded compound action potential is a summation of the individual action potentials from the different axons. In some examples, the system may also sense and record initial biphasic spikes, which are the signal artifact from the stimulator. In this disclosure, sensing the signal artifact may also be referred to as electric field imaging. When an evoked response displays resonant characteristics, e.g., resonant with the stimulation signal, the evoked biopotential may be referred to as evoked resonant neural activity, ERNA, described herein. In some examples, the resonant character of the response may occur in some locations of the patient's anatomy, but not in other locations. Implanting an intra-luminal device may be less invasive to the patient than implanting other types of electrodes.

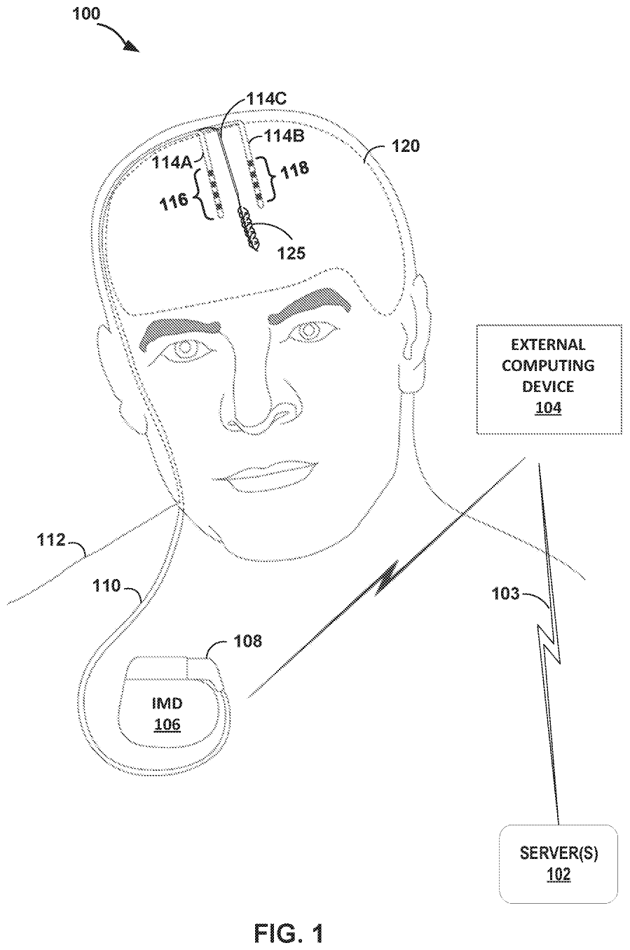

FIG. 1 is a conceptual diagram illustrating an example system 100 that includes an implantable medical device (IMD) 106 configured to deliver electrical stimulation to patient 112 and sense bioelectrical signals from the patient according to example techniques of the disclosure. The example of FIG. 1 depicts an implantable neurostimulator (INS) configured to deliver deep brain stimulation (DBS) for patient 112. However, the techniques of this disclosure may apply to other types of implantable or non-implantable electrical stimulation and sensing devices located in other areas, e.g., near the spine, pelvic area, ankle, the neck, and other locations. While some examples described in this disclosure refer to patient 112 receiving electrical stimulation therapy, in some examples, techniques described herein may apply to patients that do not receive therapy in the form of electrical stimulation. For instance, leads 114 may be implanted in a patient simply for sensing, including sensing evoked responses.

Processing circuitry of system 100 (not shown in FIG. 1), including processing circuitry within IMD 106, external computing device 104 and servers 102, in any combination may perform the functions described in this disclosure. Servers 102 may include a remote server for a cloud computing infrastructure, such as, for example a cloud or web interface.

In some examples, the delivered electrical stimulation, e.g., DBS, may be adaptive in the sense that the processing circuitry, e.g., of IMD 106 may adjust, increase, or decrease the magnitude of one or more parameters of the DBS in response to changes in patient activity or movement, a severity of one or more symptoms of a disease of the patient, a presence of one or more side effects due to the DBS, or one or more sensed signals of the patient, e.g., via intra-luminal electrode device 125.

For instance, one example of system 100 is a bi-directional DBS system with capabilities to both deliver stimulation and sense intrinsic neuronal signals. System 100 may provide for “closed-loop” therapy where IMD 106 may continuously monitor the state of certain biomarker signals and deliver stimulation according to pre-programmed routines based on the biomarker signals.

System 100 may be configured to treat a patient condition, such as a movement disorder, neurodegenerative impairment, a mood disorder, or a seizure disorder of patient 112. Patient 112 ordinarily is a human patient. In some cases, however, therapy system 100 may be applied to other mammalian or non-mammalian, non-human patients. Therapy system 100 may provide therapy to manage symptoms of patient conditions, such as, but not limited to, movement disorders and neurodegenerative impairment, seizure disorders (e.g., epilepsy) or mood (or psychological) disorders (e.g., major depressive disorder (MDD), bipolar disorder, anxiety disorders, post-traumatic stress disorder, dysthymic disorder, and obsessive-compulsive disorder (OCD)), incontinence, pain, and other symptoms.

In other examples, system 100 may be configured to deliver electrical stimulation, and based on the response the processing circuitry may receive via sensing circuitry of IMD 110, an indication of a biopotential that was evoked based on the delivered electrical stimulation. The sensed biopotential may be a measurable response indicative of a thought pattern from the patient, a neural state of the patient at the time the electrical stimulation was delivered, an indication of the function of an organ, muscle, or other tissue of the patient, and the sensed biopotential may function as one or more biomarkers.

Example therapy system 100 includes external computing device 104, IMD 106, lead extension 110, and leads 114A and 114B with respective sets of electrodes 116, 118 and lead 114C with intra-luminal electrode 125. In the example shown in FIG. 1. electrodes 116, 118 and 125 are positioned to deliver electrical stimulation to a tissue site within brain 120, such as a deep brain site under the dura mater of brain 120 of patient 112. In some examples, delivery of stimulation to one or more regions of brain 120, such as the subthalamic nucleus, globus pallidus or thalamus, may be an effective treatment to manage movement disorders, such as Parkinson's disease. Some or all of electrodes 116, 118 and 125 also may be positioned to sense neurological brain signals within brain 120 of patient 112. In some examples, some of electrodes 116, 118 and 125 may be configured to sense neurological brain signals and others of electrodes 116, 118 and 125 may be configured to deliver adaptive electrical stimulation to brain 120. In other examples, all of electrodes 116, 118 and 125 are configured to both sense neurological brain signals and deliver adaptive electrical stimulation to brain 120. In some examples, IMD 106 may be configured to deliver unipolar stimulation with at least one electrode on the housing of IMD 106.

IMD 106 includes a therapy module (e.g., which may include processing circuitry, signal generation circuitry or other electrical circuitry configured to perform the functions attributed to IMD 106, not shown in FIG. 1) that includes a stimulation generator configured to generate and deliver electrical stimulation therapy to patient 112 via a subset of any combination of electrodes 116, 118 and 125 of leads 114A, 114B, and 114C (collectively leads 114). The subset of electrodes 116, 118 and 125 that are used to deliver electrical stimulation to patient 112, and, in some cases, the polarity of the subset of electrodes 116, 118, and 125 may be referred to as a stimulation electrode combination. As described in further detail below, the stimulation electrode combination can be selected for a particular patient 112 and target tissue site (e.g., selected based on the patient condition). The group of electrodes 116, 118 includes at least one electrode and can include a plurality of electrodes. In some examples, the plurality of electrodes 116 and/or 118 may have a complex electrode geometry such that two or more electrodes are located at different positions around the perimeter of the respective lead.

In some examples, the neurological signals sensed within brain 120 may reflect changes in electrical current produced by the sum of electrical potential differences across brain tissue. Examples of neurological brain signals include, but are not limited to, bioelectric signals generated from local field potentials (LFP) sensed within one or more regions of brain 120. Electroencephalogram (EEG) signal or an electrocorticogram (ECoG) signal are also examples of bioelectric signals. For example, neurons generate the bioelectric signals, and if measured at depth, it is LFP, if measured on the cortex, it is ECoG, and if on scalp, it is EEG. In this disclosure, the term “oscillatory signal source” may be used to describe one example of a signal source that generates bioelectric signals. However, the bioelectric signals are not limited to oscillatory signals. For example, purposes, the techniques are described with oscillatory bioelectric signals from an oscillatory signal source.

In some examples, the neurological brain signals that are used to select a stimulation electrode combination may be sensed within the same region of brain 120 as the target tissue site for the electrical stimulation. As noted above, these tissue sites may include tissue sites within anatomical structures such as the thalamus, subthalamic nucleus (STN), globus pallidus (GPi) or a sinus vein, e.g., superior sagittal sinus, inferior sagittal sinus, straight sinus, sigmoid sinus, and transverse sinus of brain 120, as well as other target tissue sites. Other example target sites may include pedunculopontine nucleus (PPN), posterior subthalamic area (PSA), anterior nucleus of the thalamus (NT) and the hippocampus (HpC). The specific target tissue sites and/or regions within brain 120 may be selected based on the patient condition, e.g., epilepsy, occipital neuralgia or other headaches, and other patient conditions. Thus, in some examples, both stimulation electrode combinations and sense electrode combinations may be selected from the same set of electrodes 116, 118 and 125. In other examples, the electrodes used for delivering electrical stimulation may be different than the electrodes used for sensing neurological brain signals. In other examples, an IMD 106 configured for tibial nerve stimulation, spinal cord stimulation, vagal nerve stimulation, or other locations on patient 112 may use electrodes in a similar manner. Intraluminal electrode 125 may be located at any anatomical target, and particularly those accessed via the cerebral vasculature, for DBS and for other vasculature for other locations. In some examples, one or more intraluminal electrode devices may be located in multiple vessels near the target tissue.

Other electrode locations may include vasculature or other accessible lumens such as vasculature near the spinal cord and neck, lumens in one or more organs such as the liver, pancreas, and kidney for the renal nerve and other similar locations. In some examples, an electrode configured to be located within a lumen of an anatomical structure and proximal to target tissue of a patient may sense an ECMAP to confirm placement such as for stimulating the vagus nerve through the jugular vein. The intraluminal electrode may provide the sensing circuitry a signal from a nearby muscle response to confirm optimal location for stimulation. For example, the sternocleidomastoideonfrahyoid muscles cover the internal jugular vein (IJV) as the IJV passes under the clavicle. An ECMAP, may also be referred to as an evoked electromyography (EMG) response in this disclosure. Electromyography measures muscle response or electrical activity in response to a stimulation of the muscle.

Electrical stimulation generated by IMD 106 may be configured to manage a variety of disorders and conditions and to sense a variety of signals. In some examples, the stimulation generator of IMD 106 is configured to generate and deliver electrical stimulation as pulses to patient 112 via electrodes of a selected stimulation electrode combination. However, in other examples, the stimulation generator of IMD 106 may be configured to generate and deliver a continuous wave signal, e.g., a sine wave or triangle wave. Stimulation generation circuitry within IMD 106 (not shown in FIG. 1) may generate the electrical stimulation therapy according to a selected therapy program. In examples in which IMD 106 delivers electrical stimulation in the form of stimulation pulses, a therapy program may include a set of therapy parameter values (e.g., stimulation parameters), such as a stimulation electrode combination for delivering stimulation to patient 112, pulse frequency, pulse width, and a current or voltage amplitude of the pulses. As previously indicated, the electrode combination may indicate the specific electrodes 116, 118 and/or 125 and the respective polarities that are selected to deliver stimulation signals to tissue of patient 112 of. In some examples, the electrical stimulation generated by IMD 106 may generate, for example, burst pulses, interleaved pulses, or concurrent pulses.

In some examples, electrodes 116, 118 may be radially-segmented DBS arrays (rDBSA) of electrodes. Radially-segmented DBS arrays refer to electrodes that are segmented radially along the lead. As one example, leads 114A and 114B may include a first set of electrodes arranged circumferentially around leads 114A and 114B that are all at the same height level on leads 114A and 114B. Each of the electrodes in the first set of electrodes is a separate segmented electrode and form a level of radially-segmented array of electrodes. Leads 114A and 114B may include a second set of electrodes arranged circumferentially around leads 114A and 114B that are all at the same height level on leads 114A and 114B. Each of the electrodes in the first set of electrodes is a separate segmented electrode and form a level of radially-segmented array of electrodes. The rDBSA electrodes may be beneficial for directional stimulation and sensing.

The signal component in the beta frequency band is described as one example, and the techniques are applicable to other types of LFP activity. Furthermore, the example techniques are not limited to examples where electrodes 116, 118 are an rDBSA of electrodes. The example of using rDBSA of electrodes is described as a way of directional stimulation and sensing. However, the example techniques are also useable in examples where directional stimulation and sensing are not available or are not used. Moreover, there may be other ways of performing directional stimulation and sensing that do not require the use of an rDBSA of electrodes.

In some examples IMD 106 may generate and deliver electrical stimulation to elicit and ECAP and/or ERNA. As described above, when the evoked response displays resonant characteristics, e.g., resonant with the stimulation signal, the ECAP may be referred to as evoked resonant neural activity, ERNA. In some examples, the resonant character of the response may occur in some locations of the patient's anatomy, but not in other locations. For example, IMD 106 may sense a resonant evoked response when sensing electrodes are proximal to the STN and may not sense a resonant evoked response in some other locations.

As noted above in some examples IMD 106 may select some combination of electrodes 116 and 118 to deliver electrical stimulation and sense the evoked biopotential using intra-luminal electrode 125. In other examples, electrode 125 may both deliver the electrical stimulation and sense the evoked response. When used with an intra-luminal device, processing circuitry of system 100 may process the evoked biopotential, e.g., the ERNA, for any one or more of: Eliciting a measurable response indicative of a thought pattern or neural state that would otherwise be undetectable using a non-evoked biopotential, such as local field potential; an aid for determining the optimal location and optimal stimulation parameters for sensing biopotentials that are indicative of thought patterns or neural states; determine the location and parameters for delivering stimulation priming signals that enhance the detectability of relevant thought patterns or neural states; and assessing the patency of the intra-luminal medical device; more particularly, that the medical device is operational, in a known location, and an intimate contact with the anatomical targets of interest.

IMD 106 may be implanted within a subcutaneous pocket above the clavicle, or, alternatively, on or within cranium 122 or at any other suitable site within patient 112. Generally, IMD 106 is constructed of a biocompatible material that resists corrosion and degradation from bodily fluids. IMD 106 may comprise a hermetic housing to substantially enclose components, such as a processor, therapy module, and memory.

As shown in FIG. 1, implanted lead extension 110 is coupled to IMD 106 via connector 108 (also referred to as a connector block or a header of IMD 106). In the example of FIG. 1, lead extension 110 traverses from the implant site of IMD 106 and along the neck of patient 112 to cranium 122 of patient 112 to access brain 120. In the example shown in FIG. 1, leads 114 are implanted within the right and left hemispheres (or in just one hemisphere in some examples), respectively, of patient 112 in order to deliver electrical stimulation to one or more regions of brain 120, which may be selected based on the patient condition or disorder controlled by therapy system 100 or by the desired sensing from patient 112. The specific target tissue site and the stimulation electrodes used to deliver stimulation to the target tissue site, however, may be selected, e.g., according to the identified patient behaviors and/or other sensed patient parameters. For example, the target tissue site may be the location of the oscillatory signal source that generates the bioelectric signal having a signal component in the beta frequency band. The stimulation electrodes used to deliver stimulation to the target tissue site may be those that are most proximal to the oscillatory signal source, e.g., using the example techniques described in this disclosure. In other examples, IMD 106 may be implanted on or within cranium 122, in some examples.

Other examples of lead sets may include axial leads carrying ring electrodes disposed at different axial positions and so-called “paddle” leads carrying planar arrays of electrodes. In some examples, more complex lead array geometries may be used.

Although leads 114 are shown in FIG. 1 as being coupled to a common lead extension 110, in other examples, leads 114 may be coupled to IMD 106 via separate lead extensions or directly to connector 108. Leads 114 may be positioned to deliver electrical stimulation to or sense signals from one or more target tissue sites within brain 120. Leads 114 may be implanted to position electrodes 116, 118 at desired locations of brain 120 through respective holes in cranium 122. Leads 114 may be placed at any location within brain 120 such that electrodes 116, 118 are capable of providing electrical stimulation to target tissue sites within brain 120 during treatment. For example, electrodes 116, 118 may be surgically implanted under the dura mater of brain 120 or within the cerebral cortex of brain 120 via a burr hole in cranium 122 of patient 112, and electrically coupled to IMD 106 via one or more leads 114. As noted above, electrode 125 may be implanted via cerebral vasculature.

In the example shown in FIG. 1, electrodes 116, 118 of leads 114 are shown as ring electrodes. Ring electrodes may be used in DBS applications because ring electrodes are relatively simple to program and are capable of delivering an electrical field to any tissue adjacent to electrodes 116, 118. In other examples, electrodes 116, 118 may have different configurations. For example, at least some of the electrodes 116, 118 of leads 114 may have a complex electrode array geometry that is capable of producing shaped electrical fields. The complex electrode array geometry may include multiple electrodes (e.g., partial ring or segmented electrodes) around the outer perimeter of each lead 114, rather than one ring electrode. In this manner, electrical stimulation may be directed in a specific direction from leads 114 to enhance therapy efficacy and reduce possible adverse side effects from stimulating a large volume of tissue.

IMD 106 may include a memory (not shown in FIG. 1) to store a plurality of therapy programs that each define a set of therapy parameter values. In some examples, IMD 106 may select a therapy program from the memory based on various parameters, such as characteristics of sensed patient signals and the identified patient behaviors. IMD 106 may generate electrical stimulation based on the parameters of the selected therapy program to manage the patient symptoms or collect the desired sensed signals.

External computing device 104, which may also be referred to as external computing device 104 in this disclosure, may wirelessly communicate with IMD 106 as needed to provide or retrieve information, such as sensed biopotential recordings. A user, e.g., a clinician and/or patient 112, may use external computing device 104 to communicate with IMD 106. For example, external computing device 104 may be a clinician programmer that the clinician uses to communicate with IMD 106 and program one or more therapy programs for IMD 106. Alternatively, external computing device 104 may be a patient programmer that allows patient 112 to select programs and/or view and modify therapy parameters. The clinician programmer may include more programming features than the patient programmer. In other words, more complex or sensitive tasks may only be allowed by the clinician programmer to prevent an untrained patient from making undesirable changes to IMD 106.

When external computing device 104 is configured for use by the clinician, external computing device 104 may be used to transmit initial programming information to IMD 106. This initial information may include hardware information, such as the type of leads 114 and the electrode arrangement, the position of leads 114 within brain 120, the configuration of electrode array 116, 118, initial programs defining therapy parameter values, and any other information the clinician desires to program into IMD 106. External computing device 104 may also be capable of completing functional tests (e.g., measuring the impedance of electrodes 116, 118 of and 125 leads 114). However, in some examples, IMD 106 or external computing device 104 (, alone or in combination, may include programming instructions that when executed by processing circuitry of system 100 may automatically determine electrode configuration and therapy and sensing parameters. For example, the processing circuitry may determine which electrodes to use for stimulation based on which electrodes are most proximal to the oscillatory signal source. In other examples, the processing circuitry may determine which electrodes will elicit the desired biopotential (e.g., an ERNA or an ECAP), or to provide stimulation priming signals to target tissue of the patient that enhance the detectability of a thought pattern from patient 112. In some examples, external computing device 104 may output information indicating the selected electrode configuration for stimulation and the determined stimulation amplitude or other therapy parameter for the clinician or physician to review and confirm before IMD 106 delivers the electrical stimulation via the selected electrode configuration with the determined stimulation amplitude, pulse width and other parameters.

External computing device 104 may also be configured for use by patient 112. When configured as a patient programmer, external computing device 104 may have limited functionality (compared to a clinician programmer) in order to prevent patient 112 from altering critical functions of IMD 106 or applications that may be detrimental to patient 112. In this manner, external computing device 104 may only allow patient 112 to adjust values for certain therapy parameters or set an available range of values for a particular therapy parameter.

According to the techniques of the disclosure, a medical device (e.g., IMD 106 or external computing device 104 either alone or in combination) of system 100 may be configured to determine, based on measured signals, such as LFP activity of patient 112. when patient 112 takes medication. In some examples, the medical device of system 100 may be configured to determine a duration of when the medication is effective. external computing device 104 may display the indication of when patient 112 takes the medication, the duration of when the medication is effective, and in some examples, notification to take more medication, to facilitate a treatment for the patient on a display of external computing device 104.

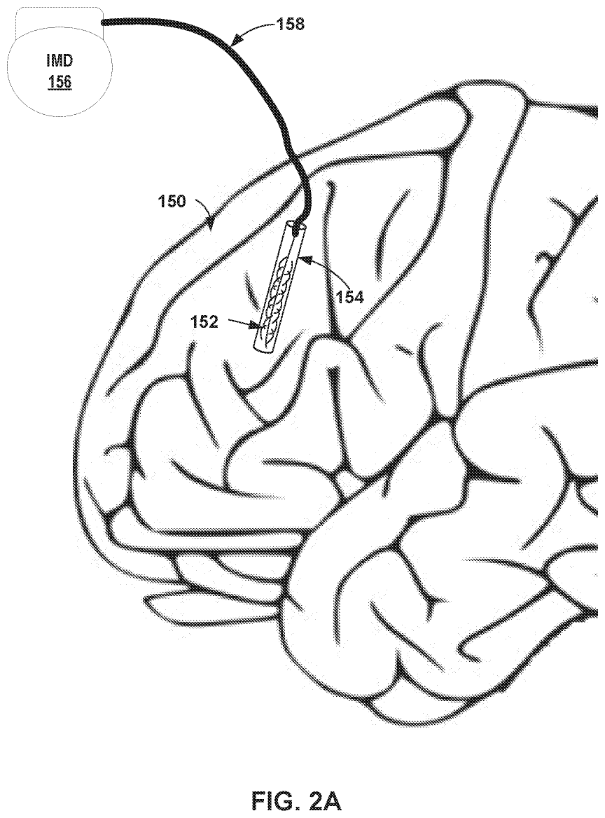

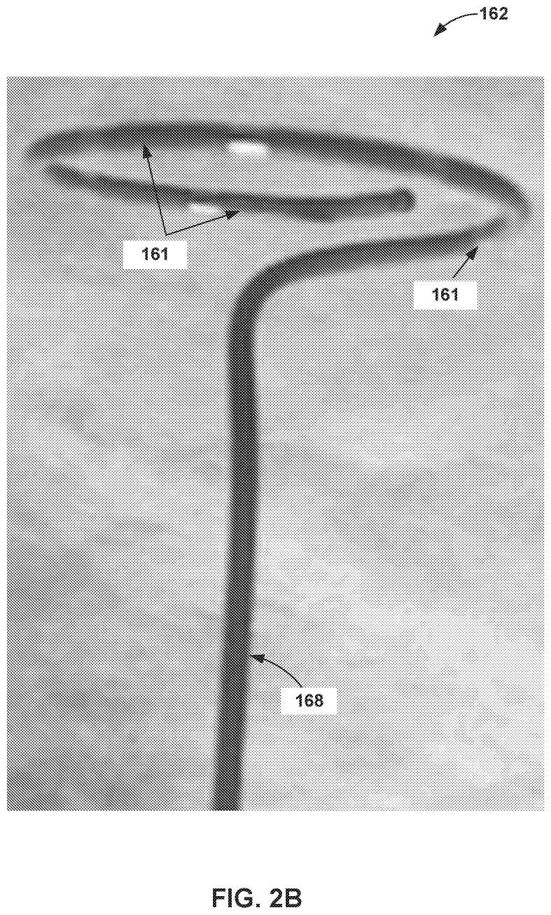

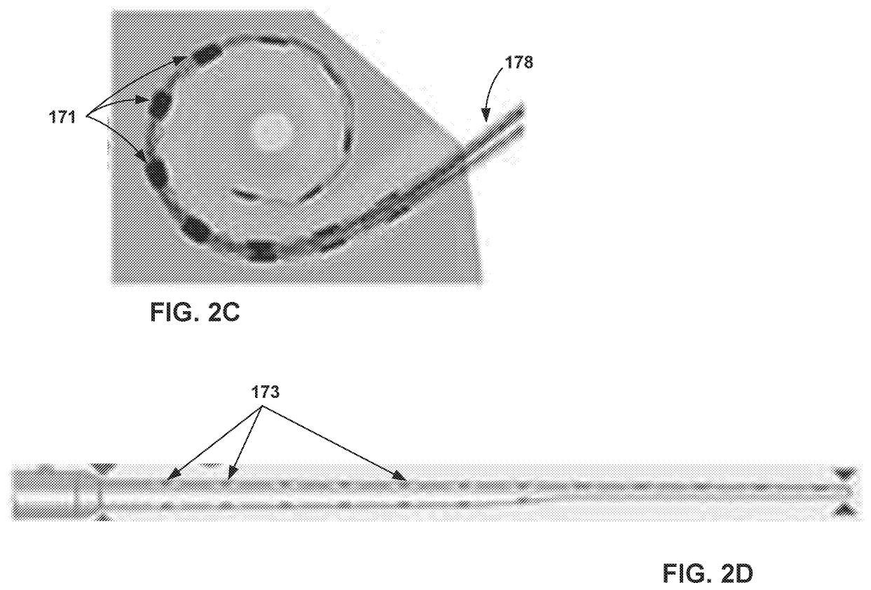

FIG. 2A is a conceptual diagram illustrating an example electrode configured to be located intra-luminally and proximal to target tissue of a patient, according to one or more techniques of this disclosure. Intra-luminal device 152 may be configured to connect to IMD 156 via lead 158 and/or a lead extension. IMD 156 may also connect to other leads and electrodes (not shown in FIG. 2) as described above in relation to FIG. 1. Intra-luminal device 152 may be arranged as an electrode array and include one or more electrodes configured to be placed with brain 150 of a patient via the cerebral vasculature, including blood vessel 154. Blood vessel 154 may be an example anatomical structure comprising a lumen and may be a vein or an artery. As described above in relation to FIG. 1, intra-luminal device 152 may also be placed in other lumens of other anatomical structures at other locations within the body of the patient. Example anatomical structures may include portions of the gastrointestinal tract, such as the esophagus or small intestine, ducts within an organ such as the pancreas or liver, or any other such anatomical structures comprising a lumen.

Electrodes of intra-luminal device 152 may be arranged on a basket or stent as shown in FIG. 2A. In some examples, one or more electrodes may be in contact with the wall of the lumen. In some examples, one or more electrodes may be in contact with fluid inside the lumen. In some examples, electrodes closer in proximity to the vessel walls, may provide improved sensing for signals generated outside the lumen. Similarly, electrodes closer in proximity, e.g., in contact with the vessel walls, may provide improved electrical stimulation conduction to nearby tissue, instead of the output signal being shunted by any fluid within the lumen, e.g., by blood, in the example of a blood vessel.

IMD 156 may be configured to deliver electrical stimulation, and sense bioelectrical signals, via intra-luminal device 152. In some examples, the invasiveness of an intraluminal sensing/stimulation system, including intra-luminal device 152 may be less than that of a system with indwelling electrodes, such as a deep-brain stimulation system 116 and 118 described above in relation to FIG. 1, implanted via a craniotomy. In some examples, biopotential signal amplitudes sensed via intra-luminal device 152 may be attenuated and stimulation coupling into the target tissue may be reduced because of the insulating effect of the body lumen walls. In other words, signals to and from target tissue must pass through the vasculature walls and therefore may be attenuated by the vasculature walls.

FIG. 2B is a conceptual diagram of an example device including one or more electrode configured to be located within a lumen of an anatomical structure and proximal to target tissue of a patient. Intraluminal device 162 may include a portion configured to connect to a lead 168, and a portion configured to hold in place within a lumen that includes one or more electrodes 161.

FIGS. 2C and 2D are a conceptual diagrams of an example device including one or more electrode configured to spiral along a lumen of an anatomical structure and proximal to target tissue of a patient. As described above, the intraluminal device of FIG. 2C may include a portion configured to connect to a lead 178, and a portion configured to hold in place within a lumen that includes one or more electrodes 171. FIG. 2D illustrates a side view of the same type of device with electrodes 173. The devices of FIGS. 2A-2D are examples of intra-luminal electrode device 125 described above in relation to FIG. 1.

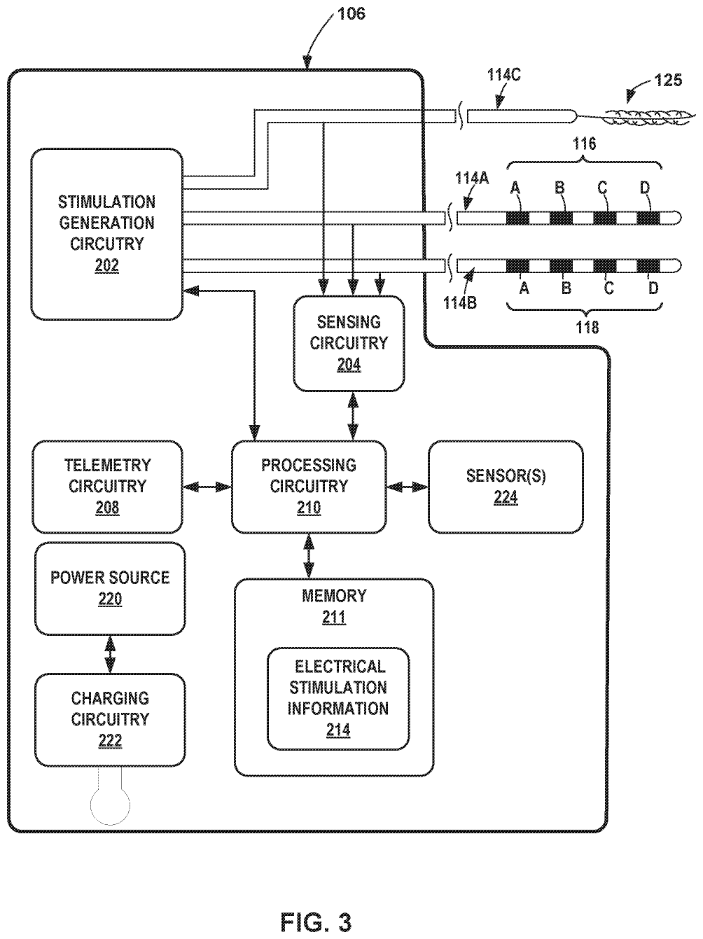

FIG. 3 is a block diagram of the example IMD 106 of FIG. 1 for delivering electrical stimulation and receiving bioelectrical signals from target tissue of a patient. In the example shown in FIG. 3, IMD 106 includes processing circuitry 210, memory 211, stimulation generation circuitry 202, sensing circuitry 204, and telemetry circuitry 208, charging circuitry 222, sensors 224 and power source 220. Each of these circuits may be or include electrical circuitry configured to perform the functions attributed to each respective circuit. Memory 211 may include any volatile or non-volatile media, such as a random-access memory (RAM), read only memory (ROM), non-volatile RAM (NVRAM), electrically erasable programmable ROM (EEPROM), flash memory, similar non-transitory computer readable storage media. Memory 211 may store computer- readable instructions that, when executed by processing circuitry 210, cause IMD 106 to perform various functions. Memory 211 may also store data, including sensed bioelectrical signals, information from accelerometers, temperature or other sensors included in sensors 224, and temporary data used for calculations or similar processing.

In the example shown in FIG. 3, memory 211 stores electrical stimulation information 214. Electrical stimulation information 214 may include program parameters (e.g., a therapy parameter set), such as a stimulation electrode combination, electrode polarity, current or voltage amplitude, pulse width, and pulse rate. In some examples, individual therapy programs may be stored as a therapy group, which defines a set of therapy programs with which stimulation may be generated. The stimulation signals defined by the therapy programs of the therapy group may be delivered together on an overlapping or non-overlapping (e.g., time-interleaved) basis.

Stimulation generation circuitry 202, under the control of processing circuitry 210, generates stimulation signals for delivery to patient 112 via selected combinations of electrodes 116, 118, and 125. Leads 114 and electrodes 116, 118, and 125 are examples of leads 114 and electrodes 116, 118, and 125 described above in relation to FIGS. 1 and 2 and have the same or similar functions and characteristics.

Processing circuitry 210 may include fixed function processing circuitry and/or programmable processing circuitry, and may comprise, for example, any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software or any combination thereof. Processing circuitry 210 may control stimulation generation circuitry 202 according to programming instructions stored in memory 211 to apply particular stimulation parameter values specified by one or more of programs, such as voltage amplitude or current amplitude, pulse width, and/or pulse rate.

In the example shown in FIG. 3, the set of electrodes 116 includes electrodes 116A, 116B, 116C, and 116D, and the set of electrodes 118 includes electrodes 118A, 118B. 118C, and 118D. Processing circuitry 210 controls stimulation generation circuitry 202 to generate and apply the stimulation signals to selected combinations of electrodes 116, 118 and 125. In some examples, processing circuitry 210 may control switch circuitry to apply the stimulation signals generated by stimulation generation circuitry 202 to selected combinations of electrodes 116, 118, and 125. In other examples, stimulation generation circuitry 202 includes a plurality of pairs of voltage sources, current sources, voltage sinks, or current sinks connected to each of electrodes 116, 118, and 125 such that each pair of electrodes has a unique signal circuit. In other words, in these examples, each of electrodes 116, 118, and 125 is independently controlled via its own signal circuit (e.g., via a combination of a regulated voltage source and sink or regulated current source and sink), as opposed to switching signals between electrodes 116, 118, and 125, and in some examples an electrode on the housing of IMD 106.

Although sensing circuitry 204 is incorporated into a common housing with stimulation generation circuitry 202 and processing circuitry 210 in FIG. 3, in other examples, sensing circuitry 204 may be in a separate housing from IMD 106 and may communicate with processing circuitry 210 via wired or wireless communication techniques. Example neurological brain signals include, but are not limited to, a signal generated from local field potentials (LFPs) within one or more regions of brain 120. EEG and ECoG signals are examples of local field potentials that may be measured within brain 120. LFPs, EEG and ECoG may be different measurements of the same bioelectric signals in the brain.

As described above in relation to FIG. 1, in some examples, IMD 106 may stimulate target tissue of the patient and sense an elicited response, such as an ERNA. In some examples, stimulation generation circuitry may output electrical stimulation via some combination of electrodes 116 and 118 and sense the response via intra-luminal electrode 125. In other examples, intra-luminal electrode 125, may provide electrical stimulation, e.g., source or sink current, and also sense the elicited response. As described above in relation to FIG. 2, in some examples, intra-luminal electrode 125 may be arranged as an array and include a plurality of electrodes.

One application is that IMD 106 may be configured to deliver stimulation configured to elicit a measurable response indicative of a thought pattern or neural state that would otherwise be undetectable using a non-evoked biopotential, such as a local field potential. Processing circuitry 210, and/or other processing circuitry of system 100 depicted in FIG. 1, may iteratively select electrodes to deliver stimulation while recording an evoked response such as ECAPs or ERNAs from electrodes not used for stimulation.

Sensing circuitry 204 may measure one or more characteristics of the biopotential, such as the ERNA, while the patient is both engaged and not engaged in a particular thought pattern. Some examples of measured characteristics may include an amplitude, latency, area under a curve, morphology, number of zero crossings, resonant frequency, decay constant (e.g., time for the ERNA signal to decay), or number of maxima and maxima within the signal. In some examples, IMD 106 may sense changes in biopotentials during patient movement or lack of movement, e.g., changes in an ERNA. In some examples, processing circuitry 210 may correlate signals from the one or more accelerometers of sensors 224 of sensed position and/or movement to the evoked biopotentials.

In some examples, the processing circuitry may be trained to differences, which in some examples may be specific for a specific patient. For example, training may include the difference between a baseline and a specified thought pattern or specified neural state. Other differences may include the difference between a first neural state and a second neural state, or between two or more different thought patterns. Once trained, the processing circuitry may detect a neural state change and determine, based on that neural state change, that a trigger event has occurred, such as activation/inhibition of an indicator, a system state change, a telemetry event, closed loop adjustments to electrical stimulation therapy, or other actions.

In some examples, processing circuitry 210 may not be able to detect a desired thought pattern or neural state using local field potentials alone. An active approach like detecting evoked biopotentials, such as ECAPs/ERNAs, may elicit the neural state changes indicative of a thought pattern change. In some examples, processing circuitry 210 may first determine that the neural state change is undetectable with LFPs, then apply the active approach to elicit and measure ERNA. In other examples, processing circuitry 210 may use an accelerometer signal as trigger to then apply the active approach and elicit an evoked biopotential.

Processing circuitry, e.g., processing circuitry 210 of IMD 106, may also analyze sensed evoked biopotentials as an aid for determining an optimal location for sensing biopotentials that are indicative of thought patterns or neural states. In some examples, the LFPs detectable within a body lumen that manifest with thought pattern or neural state changes may be very low in amplitude and require extensive signal processing or lengthy periods of observation. However, evoked biopotentials may have a larger. more detectable amplitude because of the synchronous and compound activation of the neural circuits implicated with the neural state change. Accordingly, IMD 106 may output electrical stimulation to elicit evoked biopotentials and analyze the received sensed evoked biopotentials to assess which electrodes and electrode locations may be best suited for LFP detection. In some examples, a clinician may perform steps of this type of assessment during device fitting and training of IMD 106 to a patient.

In some examples, the processing circuitry may deliver stimulation priming signals that enhance the detectability of relevant thought patterns or neural states. As noted above, the LFPs relevant to thought pattern or neural state changes are often difficult to detect within a body lumen. In some examples, IMD 106 may output neurostimulation via some combination of electrodes 116, 118 and 125 to “prime” the neural circuits involved in the thought pattern or neural state changes. In some examples, the neurostimulation, e.g., electrical stimulation signals with certain parameters, may make the neural target more susceptible to activation and subsequently generate larger LFPs when a neural state change occurs. In some examples, the output neurostimulation may be delivered as bursts, e.g., to save power or avoid undesirable effects such neural habituation. IMD 106 may use the sensed evoked biopotentials to monitor the neural target and ensure the priming neurostimulation has been delivered. As described above, selecting the electrical stimulation parameters, and electrode combinations, may be set up during fitting and training.

In other examples, IMD 106 may assess the patency of the intra-luminal medical device, e.g., intra-luminal electrode 125. Some example criteria for patency may include that electrode 125 is operational, is in a known location, and is in contact with the anatomical targets of interest, e.g., such that electrode 125 may deliver stimulation, or sense signals from the target tissue. Because some of the biosignals of interest may be challenging to detect, as noted above, determining whether a signal is truly not present, or that electrode 125 not operating properly or in an incorrect location may be difficult. Unless otherwise noted the terms “biosignals,” bipotential, bioelectrical signals and similar terms may be used interchangeably. In this disclosure, electrical stimulation delivered to evoke a response may be different (e.g., delivered with different parameters such as amplitude and pulse width) than electrical stimulation therapy delivered to manage a patient's symptoms.

IMD 106 may be configured to use delivered electrical stimulation to evoke a known neural response, e.g., an evoked biopotential such as an ERNA in the target tissue. A degraded or absent evoked response may indicate that the system is not operating correctly or is that electrode 135 is in an incorrect location. Conversely, processing circuitry of IMD 106 may determine that electrode 125 is operating properly and/or in a desired location proximate to the target tissue based on sensing an evoked biopotential, for example, an ECAP in response to a control pulse configured to elicit the ECAP.

Telemetry circuitry 208 supports wireless communication between IMD 106 and an external computing device 104 or another computing device under the control of processing circuitry 210. Processing circuitry 210 of IMD 106 may receive, as updates to programs, values for various stimulation parameters such as magnitude and electrode combination, from external computing device 104 via telemetry circuitry 208. The updates to the therapy programs may be stored within therapy programs 214 portion of memory 211. Telemetry circuitry 208 in IMD 106, as well as telemetry modules in other devices and systems described herein, such as external computing device 104, may accomplish communication by radiofrequency (RF) communication techniques. In addition, telemetry circuitry 208 may communicate with external medical device external computing device 104 via proximal inductive interaction of IMD 106 with external computing device 104. Accordingly, telemetry circuitry 208 may send information to external computing device 104 on a continuous basis, at periodic intervals, or upon request from IMD 106 or external computing device 104.

In some examples, processing circuitry 210 may continuously measure the one or more bioelectric signals in real time. In other examples, processing circuitry 210 may periodically sample the one or more bioelectric signals according to a predetermined frequency or after a predetermined amount of time. In some examples, processing circuitry 210 may periodically sample the signal at a frequency of approximately 150 Hertz.

Power source 220 delivers operating power to various components of IMD 106. Power source 220 may include a small rechargeable or non-rechargeable battery and a power generation circuit to produce the operating power. Recharging may be accomplished through proximal inductive interaction between an external charger and an inductive charging coil, e.g., connected to charging circuitry 222 within IMD 106. In some examples, power requirements may be small enough to allow IMD 106 to utilize patient motion and implement a kinetic energy-scavenging device to trickle charge a rechargeable battery. In other examples, traditional batteries may be used for a limited period of time.

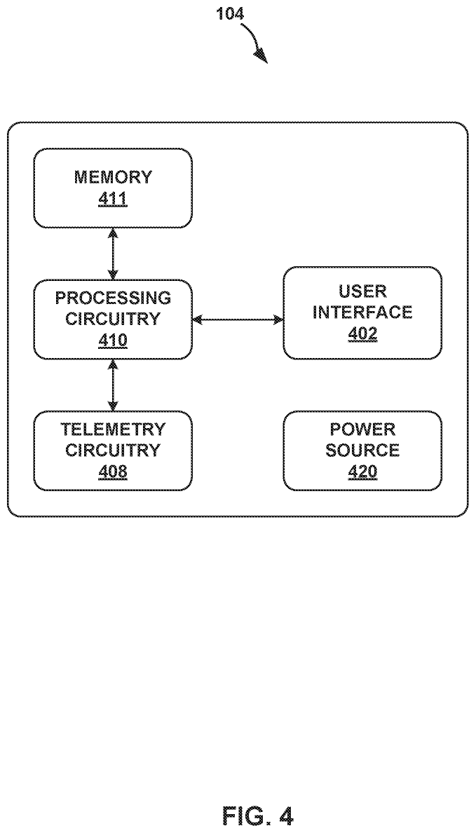

FIG. 4 is a block diagram of the external computing device 104 of FIG. 1. Although external computing device 104 may generally be described as a hand-held device, external computing device 104 may be a larger portable device or a more stationary device. In addition, in other examples, external computing device 104 may be included as part of an external charging device or include the functionality of an external charging device. As illustrated in FIG. 4, external computing device 104 may include processing circuitry 410, memory 411, user interface 402, telemetry circuitry 408, and power source 420. Memory 411 may store instructions that, when executed by processing circuitry 410, cause processing circuitry 410 and external computing device 104 to provide the functionality ascribed to external computing device 104 throughout this disclosure. Each of these components, or modules, may include electrical circuitry that is configured to perform some, or all of the functionality described herein. In some examples, processing circuitry 410, combination with processing circuitry 210 of FIG. 3, may perform some of the functions described above in relation to FIGS. 1, 2, and 3, such as analyzing and responding to an evoked response sensed by intra-luminal electrode 125.

In general, external computing device 104 comprises any suitable arrangement of hardware, alone or in combination with software and/or firmware, to perform the techniques attributed to external computing device 104, and processing circuitry 410, user interface 402, and telemetry circuitry 408 of external computing device 104. In various examples, external computing device 104 may include one or more processors, which may include fixed function processing circuitry and/or programmable processing circuitry, as formed by, for example, one or more microprocessors, DSPs, ASICs, FPGAs, or any other equivalent integrated or discrete logic circuitry, as well as any combinations of such components. External computing device 104 also, in various examples, may include a memory 411, such as RAM, ROM, PROM, EPROM, EEPROM, flash memory, a hard disk, a CD-ROM, comprising executable instructions for causing the one or more processors to perform the actions attributed to them. Moreover, although processing circuitry 410 and telemetry circuitry 408 are described as separate modules, in some examples, processing circuitry 410 and telemetry circuitry 408 may be functionally integrated with one another. In some examples, processing circuitry 410 and telemetry circuitry 408 correspond to individual hardware units, such as ASICs, DSPs, FPGAs, or other hardware units.

Memory 411 (e.g., a storage device) may store instructions that, when executed by processing circuitry 410, cause processing circuitry 410 and external computing device 104 to provide the functionality ascribed to external computing device 104 throughout this disclosure. For example, memory 411 may include instructions that cause processing circuitry 410 to obtain a parameter set from memory or receive a user input and send a corresponding command to IMD 106, or instructions for any other functionality. In addition, memory 411 may include a plurality of programs, where each program includes a parameter set that defines stimulation therapy.

User interface 402 may include a button or keypad, lights, a speaker for voice commands, a display, such as a liquid crystal (LCD), light-emitting diode (LED), or organic light-emitting diode (OLED). In some examples the display may be a touch screen. User interface 402 may be configured to display any information related to the delivery of stimulation therapy, identified patient behaviors, sensed patient parameter values, patient behavior criteria, displaying detected thought patterns, identified neural activity, efficacy of the placed intra-luminal electrode device or any other such information. User interface 402 may also receive user input. The input may be, for example, in the form of pressing a button on a keypad or selecting an icon from a touch screen.

Telemetry circuitry 408 may support wireless communication between IMD 106 and external computing device 104 under the control of processing circuitry 410. Telemetry circuitry 408 may also be configured to communicate with another computing device via wireless communication techniques, or direct communication through a wired connection. In some examples, telemetry circuitry 408 provides wireless communication via an RF or proximal inductive medium. In some examples, telemetry circuitry 408 includes an antenna, which may take on a variety of forms, such as an internal or external antenna.

Examples of local wireless communication techniques that may be employed to facilitate communication between external computing device 104 and IMD 106 include RF communication according to the 802.11 or Bluetooth specification sets or other standard or proprietary telemetry protocols. In this manner, other external devices may be capable of communicating with external computing device 104 without needing to establish a secure wireless connection. In some examples, external computing device may communicate via inductive coils, during or interleaved with recharging IMD 106, such as a coil connected to charging circuitry 222 of FIG. 3.

In some examples, processing circuitry 410 of external computing device 104 defines the parameters of electrical stimulation therapy, stored in memory 411, for delivering adaptive DBS to patient 112. In one example, processing circuitry 410 of external computing device 104, via telemetry circuitry 408, issues commands to IMD 106 causing IMD 106 to deliver electrical stimulation therapy via electrodes 116, 118 and 125 via leads 114, as described above in relation to FIGS. 1-3.

External computing device 104 may also configure which electrodes that IMD 106 uses to deliver electrical stimulation, e.g., to make a neural target more susceptible to activation and subsequently generate larger LFPs when a neural state change occurs. Similarly, external computing device 104 may configure IMD 106 to elicit a measurable response indicative of a thought pattern or neural state that would otherwise be undetectable using a non-evoked biopotential.

According to the techniques of the disclosure, external computing device 104 may be configured to determine, e.g., based on LFP activity of patient 112, when patient 112 takes medication. In some examples, external computing device 104 may be configured to determine, based on LFP activity of patient 112, a duration of when the medication is effective. External computing device 104 may be configured to determine, based on LFP activity of patient 112, both when patient 112 takes medication and a duration of when the medication is effective. External computing device 104 may output an indication of when patient 112 takes the medication and/or the duration of when the medication is effective to facilitate a treatment for the patient.

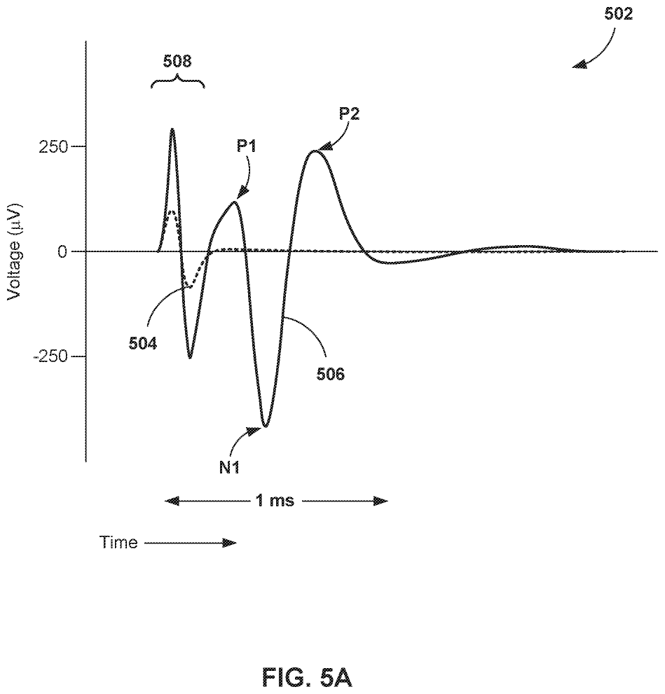

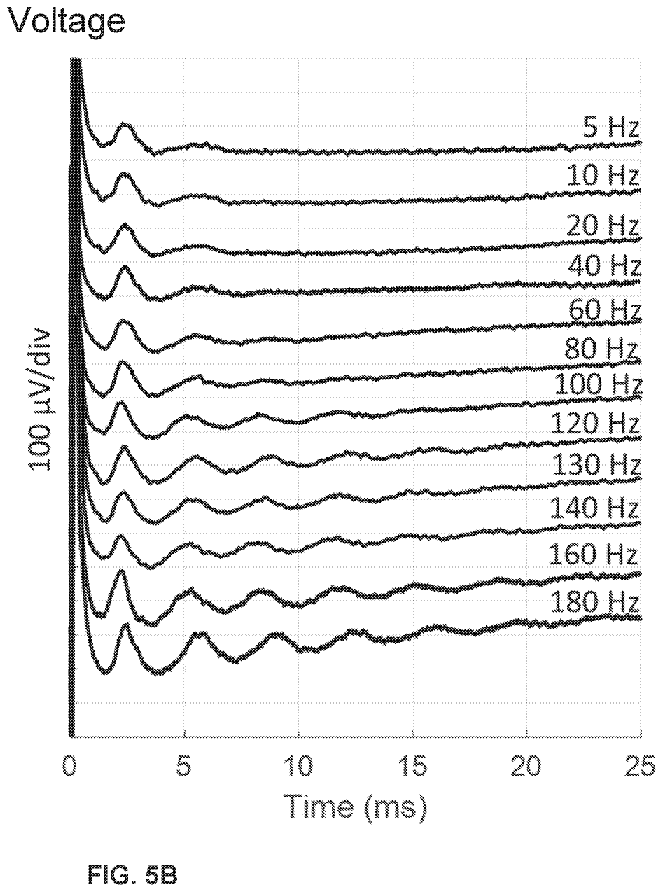

FIG. 5A is a time graph of example evoked compound action potentials (ECAPs) sensed for respective stimulation pulses, in accordance with one or more techniques of this disclosure. As shown in FIG. 4, graph 502 shows example ECAP signal 504 (dotted line) and ECAP signal 506 (solid line). In some examples, each of ECAP signals 504 and 506 are sensed from stimulation pulses (e.g., a control pulse) that were delivered from a guarded cathode, where the stimulation pulses are bi-phasic pulses including an interphase interval between each positive and negative phase of the pulse. In some such examples, the guarded cathode includes stimulation electrodes located at the end of an 8-electrode lead (e.g., leads 114 of FIGS. 1 and 3) while two sensing electrodes may be located at the other end of the 8-electrode lead. ECAP signal 504 illustrates the voltage amplitude sensed as a result from a sub-detection threshold stimulation pulse, or a stimulation pulse which results in no detectable ECAP. In other examples, not shown in FIG. 5A, the IMD may output a monophasic, tri-phasic, or pulses with another quantity of phases to elicit an evoked response.

Sensing circuitry of an IMD, e.g., IMD 106 of FIGS. 1 and 3, may detect peaks 508 of ECAP signal 504. Peaks 508 represent stimulation signals of the delivered stimulation pulse. However, no propagating signal is detected after the stimulation signal in ECAP signal 504 because the stimulation pulse had an intensity (e.g., an amplitude and/or pulse width) that was “sub-threshold” or below a detection threshold (e.g., a sub-detection threshold) and/or below a propagation threshold (e.g., a sub-propagation threshold).

In contrast to ECAP signal 504, ECAP signal 506 represents the voltage amplitude detected from a supra-detection stimulation threshold stimulation pulse. Peaks 508 of ECAP signal 506 are detected and represent stimulation signals of the delivered stimulation pulse. After peaks 508, ECAP signal 506 also includes peaks P1, N1, and P2, which are three typical peaks representative of propagating action potentials from an ECAP. The example duration of the stimulation signal and peaks P1, N1, and P2 is approximately 1 millisecond (ms).

When detecting the ECAP of ECAP signal 506, different characteristics may be identified. For example, the characteristic of the ECAP may be the amplitude between N1 and P2. This N1-P2 amplitude may be detectable even if the stimulation signal impinges on P1, a relatively large signal, and the N1-P2 amplitude may be minimally affected by electronic drift in the signal. In other examples, the characteristic of the ECAP used to control subsequent stimulation pulses (e.g., control pulses and/or informed pulses) may be an amplitude of P1, N1, or P2 with respect to neutral or zero voltage. In some examples, the characteristic of the ECAP used to control subsequent stimulation pulses is a sum of two or more of peaks P1, N1, or P2. In other examples, the characteristic of ECAP signal 406 may be the area under one or more of peaks P1, N1, and/or P2. In other examples, the characteristic of the ECAP may be a ratio of one of peaks P1, N1, or P2 to another one of the peaks. In some examples, the characteristic of the ECAP is a slope between two points in the ECAP signal, such as the slope between N1 and P2. In other examples, the characteristic of the ECAP may be the time between two points of the ECAP, such as the time between N1 and P2.

The time between when the stimulation pulse is delivered and a point in the ECAP signal may be referred to as a latency of the ECAP and may indicate the types of fibers being captured by the stimulation pulse (e.g., a control pulse). ECAP signals with lower latency (i.e., smaller latency values) indicate a higher percentage of nerve fibers that have faster propagation of signals, whereas ECAP signals with higher latency (i.e., larger latency values) indicate a higher percentage of nerve fibers that have slower propagation of signals. Latency may also refer to the time between an electrical feature is detected at one electrode and then detected again at a different electrode. This time, or latency, is inversely proportional to the conduction velocity of the nerve fibers. Other characteristics of the ECAP signal may be used in other examples.

The amplitude of the ECAP signal increases with increased amplitude of the stimulation pulse, as long as the pulse amplitude is greater than threshold such that nerves depolarize and propagate the signal. The target ECAP characteristic (e.g., the target ECAP amplitude) may be determined from the ECAP signal detected from a stimulation pulse (or a control pulse) when informed pulses are determined to deliver effective therapy to patient 105. The ECAP signal thus is representative of the distance between the stimulation electrodes and the nerves appropriate for the stimulation parameter values of the informed pulses delivered at that time. Therefore, IMD 106, of FIG. 1, may attempt to use detected changes to the measured ECAP characteristic value to change therapy pulse parameter values and maintain the target ECAP characteristic value during therapy pulse delivery.

FIG. 5B is a time graph illustrating an example evoked resonant neural activity (ERNA) received by an intra-luminal medical device according to an example of the techniques of the disclosure. To elicit an evoked response, the electrical stimulation generator output a pulse for frequencies less than 60 Hz or burst of pulses for frequencies greater than 60 Hz. Time zero in the example of FIG. 5B is immediately after the delivered control pulse. For frequencies greater than 60 Hz, zero time is after the last pulse of the burst of pulses. The pulse widths for the example of FIG. 5B were approximately 120 microseconds (μs).

In some examples, the response shown in the example of FIG. 5B may be sensed when stimulation and the sense electrodes are closest to STN target. In some examples such a response may also be sensed when the stimulation and sense electrode contacts are the same (i.e., the stim and sense electrode contacts do not necessarily have to be different electrodes).

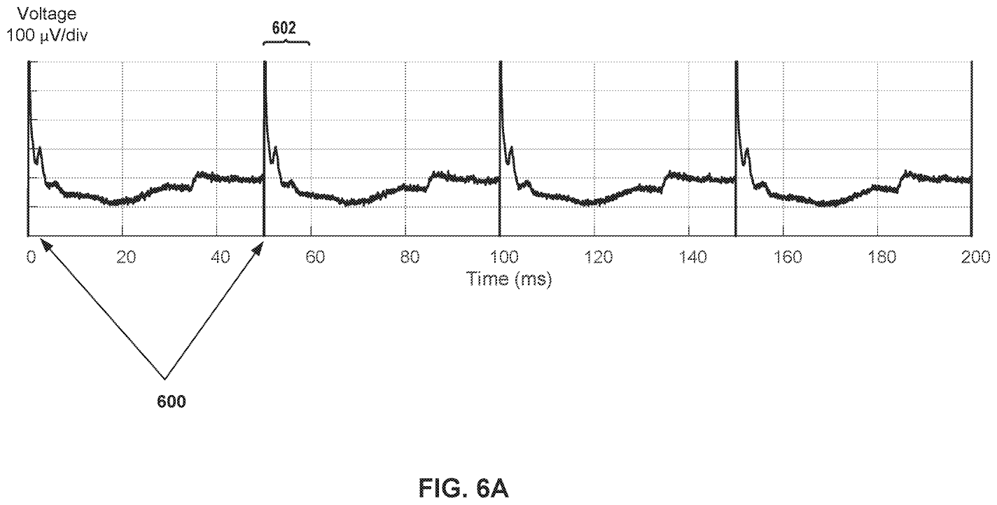

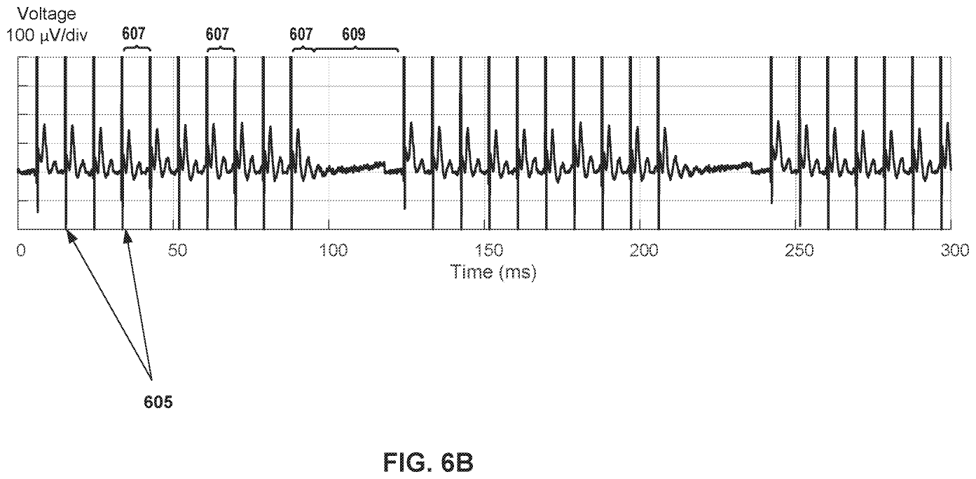

For lower frequencies, e.g., less than 40 Hz, the sensed evoked response appears similar to an ECAP, as described above in relation to FIG. 5A. In some examples, the evoked biopotentials may have different characteristics depending on the electrode location. For example, a difference for ECAP in brain may include that the time constant is different than the SCS example shown in FIG. 5A. The response in FIG. 6A may be representative of DBS ECAP from lower, 20Hz stimulation. As the frequency increases, the example of FIG. 5B shows an increase in resonance in the sensed evoked response. In other words, the sensed evoked response may include additional peaks and troughs from the resonance of the neural target connectivity. In this manner, to elicit a resonant evoked response, it may be desirable to stimulate the target tissue with electrical stimulation of higher frequencies.

FIGS. 6A and 6B are time graphs illustrating an example of resonant bioelectrical signals (e.g., an ERNA) evoked from electrical stimulation, according to one or more techniques of this disclosure. FIG. 6A is a time graph of a sensed evoked response to 20 Hz stimulation. The sensed evoked response for each pulse 602 includes peaks and troughs similar to an ECAP, as described above in relation to FIG. 5A. In some examples, the evoked biopotential for such electrical stimulation has features similar to the ECAP elicited for electrodes implanted proximal to the spinal cord, e.g., for spinal cord stimulation (SCS).

FIG. 6B is a time graph illustrating a sensed evoked response to three consecutive bursts of 110 Hz stimulation. Each pulse 605 evokes a response 607, but because the period is about 90 milliseconds (ms), then the sensing circuitry of the IMD may not have enough time to record the peaks and troughs of the resonant activity 609. Therefore, the processing circuitry of the IMD may be programmed to pause stimulation periodically (e.g., cease stimulation for a predetermined period of time) to measure and analyze characteristics of the resonant response. Some examples of characteristics that the IMD may measure and analyze include the resonant frequency, e.g., of the peaks and troughs, or some subset of peaks and troughs, amplitude, damping of the resonance, latency changes of the peaks and troughs and other similar characteristics, phase alignment of evoked response with underlying resonance. In some examples analysis of sensed signals may include trends of one or more selected characteristics of sensed waveform, e.g., the amplitude of the evoked response changes indicating a change in neural state, or the latency of peak and troughs shifts (thus changing the resonant frequency) which could indicate change in neural state. In some examples, the number of pulses in a burst that elicits the resonant activity may depend on various factors including individual patient physiology, neural state, selected medication, asleep vs. awake, disease progression electrode location, selected burst frequency, and other factors. In other examples, measured characteristics of ERNA response may could indicate need to adjust stimulation parameters such as stimulation frequency, amplitude, pulse width and other parameters. In some examples, processing circuitry 210 of FIG. 3 may adjust stimulation parameters in response to measured characteristics of evoked biopotentials.

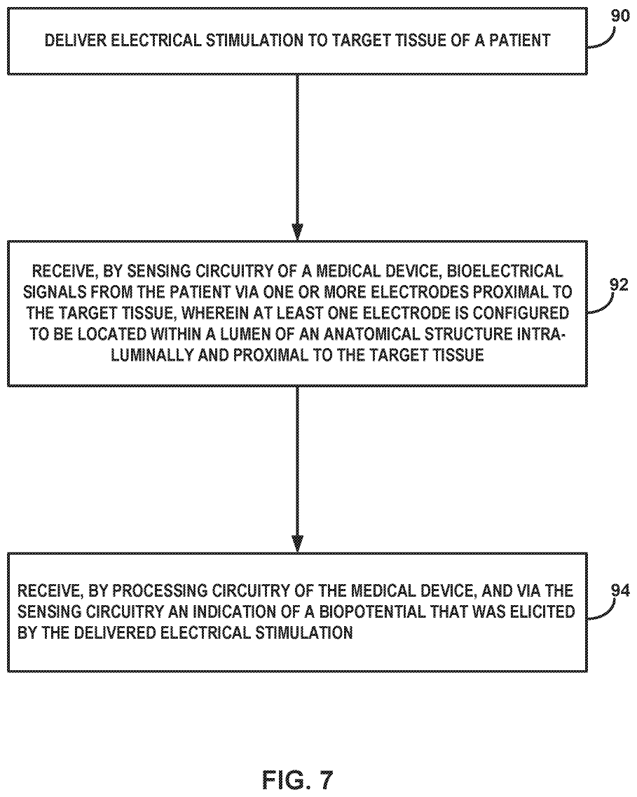

FIG. 7 is a flow chart illustrating an example mode of operation of a system of this disclosure. The system described by FIG. 7 includes system 100, described above in relation to FIG. 1.

A medical device, such as IMD 106 of FIG. 1 may deliver electrical stimulation to target tissue of a patient (90). Target tissue may include brain tissue, as depicted in FIGS. 1 and 2, or other target tissue such as spinal cord nerves, tibial nerves, internal organs, muscle tissue and other patient tissue. Stimulation generation circuitry 202 may generate the stimulation electrical stimulation defined by one or more parameters and based on control signals from processing circuitry 210 of FIG. 3.

Sensing circuitry of the medical device, such as sensing circuitry 204 of FIG. 3. may receive bioelectrical signals from the patient via one or more electrodes 116, 118 proximal to the target tissue (92). In some examples, at least one electrode 125 is configured to be located within a lumen of an anatomical structure and proximal to the target tissue, as shown in FIGS. 1, 2 and 3.

Processing circuitry 210 of medical device 106 of FIG. 2, may receive via sensing circuitry 204, an indication of a biopotential that was elicited by the delivered electrical stimulation (94). As described in detail above, the sensed biopotential may be a measurable response indicative of at least one of a thought pattern from the patient or of a neural state of the patient at the time the electrical stimulation was delivered.

The techniques of this disclosure may also be described in the following examples.

Example 1: A system comprising: an electrode configured to be located within a lumen of an anatomical structure and proximal to target tissue of a patient; a medical device comprising: electrical stimulation circuitry configured to deliver electrical stimulation to the patient; sensing circuitry configured to detect bioelectrical signals from the patient via the electrode; and processing circuitry configured to: control the electrical stimulation circuitry to deliver electrical stimulation to the patient; receive, via the sensing circuitry, an indication of a biopotential that was elicited by the delivered electrical stimulation, wherein the sensed biopotential is a measurable response indicative of at least one of: a thought pattern from the patient; or a neural state of the patient at the time the electrical stimulation was delivered.

Example 2: The system of example 1, comprising a plurality of electrodes separate from the electrode configured to be located within the lumen of the anatomical structure, wherein the electrical stimulation circuitry is configured to deliver the electrical stimulation therapy via one or more of the plurality of electrodes.

Example 3: The system of any of examples 1 and 2, wherein the delivered electrical stimulation elicits a biopotential comprising an evoked resonant neural activity (ERNA) from the patient.

Example 4: The system of example 3, wherein the target tissue of the patient comprises a location within the patient's subthalamic nucleus (STN).

Example 5: The system of any of examples 3 and 4, wherein a parameter that at least partially defines the electrical stimulation comprises a stimulation frequency, wherein the elicited ERNA from the patient exhibits one or more characteristics, wherein the one or more characteristics comprise a resonant frequency of measured peaks and troughs of the ERNA.

Example 6: The system of any of examples 1 through 5, wherein the delivered electrical stimulation elicits a biopotential comprising an evoked compound action potential (ECAP) from the patient.

Example 7: The system of any of examples 1 through 6, wherein the delivered electrical stimulation is configured to provide stimulation priming signals to target tissue of the patient that enhance the detectability of at least one of: the thought pattern from the patient; or the neural state of the patient at the time the electrical stimulation was delivered.

Example 8: The system of any of examples 1 through 7, wherein the processing circuitry is configured to adjust one or more parameters that define the delivered electrical stimulation based on the received biopotential, wherein the received biopotential is indicative of a location of target tissue of the patient for sensing biopotentials that are indicative of thought patterns or neural states.

Example 9: The system of any of examples 1 through 8, wherein the processing circuitry is configured to adjust one or more parameters that define the delivered electrical stimulation based on the received biopotential, wherein the received biopotential is indicative of the patency of the electrode located within the lumen of the anatomical structure.

Example 10: The system of any of examples 1 through 9, wherein the processing circuitry is configured to adjust one or more parameters that define the delivered electrical stimulation based on the received biopotential, wherein the received biopotential is indicative of one or more of: the electrode is operational, the electrode is in a known location, and the electrode is in contact with an anatomical target of interest of the patient.

Example 11: The system of any of examples 1 through 10, wherein the anatomical structure is a blood vessel located in the brain.

Example 12: The system of any of examples 1 through 11, wherein the electrical stimulation circuitry is configured to deliver the electrical stimulation to the patient via the electrode configured to be located within the lumen of the anatomical structure.

Example 13: A method comprising: delivering electrical stimulation to target tissue of a patient; receiving, by sensing circuitry of a medical device, bioelectrical signals from the patient via one or more electrodes proximal to the target tissue, wherein at least one electrode is configured to be located within a lumen of an anatomical structure and proximal to the target tissue; receiving, by processing circuitry, and via the sensing circuitry an indication of a biopotential that was elicited by the delivered electrical stimulation, wherein the sensed biopotential is a measurable response indicative of at least one of: a thought pattern from the patient; or a neural state of the patient at the time the electrical stimulation was delivered.

Example 14: The method of example 13, wherein the medical device is an implantable medical device.

Example 15: The method of any of examples 13 and 14, wherein the medical device comprises the processing circuitry, the method further comprising, controlling, by the processing circuitry, electrical stimulation circuitry of the medical device to deliver the electrical stimulation to the patient.

Example 16: The method of example 15, further comprising controlling, by the processing circuitry, the electrical stimulation circuitry to deliver the electrical stimulation to the patient via the electrode configured to be located within the lumen of the anatomical structure.

Example 17: The method of any of examples 15 and 16, further comprising controlling, by the processing circuitry, the electrical stimulation circuitry to deliver the electrical stimulation to the patient via a plurality of electrodes configured to be separate from the electrode located within the lumen of the anatomical structure.

Example 18: The method of any of examples 13 through 17, wherein the sensed biopotential is evoked resonant neural activity (ERNA) from the patient.

Example 19: The method of example 18, wherein the elicited ERNA from the patient exhibits one or more characteristics, and wherein the one or more characteristics comprise a resonant frequency of measured peaks and troughs of the ERNA.

Example 20: A medical device comprising: electrical stimulation circuitry configured to deliver electrical stimulation to a patient via one or more electrodes, wherein at least one electrode is configured to be located within a lumen of an anatomical structure and proximal to target tissue of the patient; sensing circuitry configured to detect bioelectrical signals from the patient via the electrode configured to be located intra- luminally; and processing circuitry configured to: control the electrical stimulation circuitry to deliver electrical stimulation to the patient; receive via the sensing circuitry an indication of a biopotential that was elicited by the delivered electrical stimulation, wherein the sensed biopotential is a measurable response indicative at least one of: a thought pattern from the patient; or a neural state of the patient at the time the electrical stimulation was delivered.

In one or more examples, the functions described above may be implemented in hardware, software, firmware, or any combination thereof. For example, the various components of FIGS. 3 and 4, such as processing circuitry 210, processing circuitry 410, telemetry circuitry 408 and charging circuitry 222 may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

The term “non-transitory” may indicate that the storage medium is not embodied in a carrier wave or a propagated signal. In certain examples, a non-transitory storage medium may store data that can, over time, change (e.g., in RAM or cache). By way of example, and not limitation, such computer-readable storage media, may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may include one or more computer-readable storage media.

Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Combinations of the above should also be included within the scope of computer-readable media.

Instructions may be executed by one or more processors, such as one or more DSPs. general purpose microprocessors, ASICs, FPGAs, or other equivalent integrated or discrete logic circuitry. Accordingly, the term “processor” and “processing circuitry,” as used herein, may refer to any of the foregoing structure or any other structure suitable for implementation of the techniques described herein. Also, the techniques could be fully implemented in one or more circuits or logic elements.

The techniques of this disclosure may be implemented in a wide variety of devices or apparatuses, including, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

The following examples are a non-limiting list of clauses in accordance with one or more techniques of this disclosure.

Various examples of the disclosure have been described. These and other examples are within the scope of the following claims.

Example 1. A system comprising: an electrode configured to be located within a lumen of an anatomical structure and proximal to target tissue of a patient; a medical device comprising: electrical stimulation circuitry configured to deliver electrical stimulation to the patient; sensing circuitry configured to detect bioelectrical signals from the patient via the electrode; and processing circuitry configured to: control the electrical stimulation circuitry to deliver electrical stimulation to the patient; receive, via the sensing circuitry, an indication of a biopotential that was elicited by the delivered electrical stimulation, wherein the sensed biopotential is a measurable response indicative of at least one of: a thought pattern from the patient; or a neural state of the patient at the time the electrical stimulation was delivered.

Example 2. The system of Example 1, comprising a plurality of electrodes separate from the electrode configured to be located within the lumen of the anatomical structure, wherein the electrical stimulation circuitry is configured to deliver the electrical stimulation therapy via one or more of the plurality of electrodes.

Example 3. The system of Example 1, wherein the delivered electrical stimulation elicits a biopotential comprising an evoked resonant neural activity (ERNA) from the patient.

Example 4. The system of Example 3, wherein the target tissue of the patient comprises a location within the patient's subthalamic nucleus (STN).

Example 5. The system of Example 3, wherein a parameter that at least partially defines the electrical stimulation comprises a stimulation frequency, wherein the elicited ERNA from the patient exhibits one or more characteristics, wherein the one or more characteristics comprise a resonant frequency of measured peaks and troughs of the ERNA.

Example 6. The system of Example 1, wherein the delivered electrical stimulation elicits a biopotential comprising an evoked compound action potential (ECAP) from the patient.

Example 7. The system of Example 1, wherein the delivered electrical stimulation is configured to provide stimulation priming signals to target tissue of the patient that enhance the detectability of at least one of: the thought pattern from the patient; or the neural state of the patient at the time the electrical stimulation was delivered.

Example 8. The system of Example 1, wherein the processing circuitry is configured to adjust one or more parameters that define the delivered electrical stimulation based on the received biopotential, wherein the received biopotential is indicative of a location of target tissue of the patient for sensing biopotentials that are indicative of thought patterns or neural states.

Example 9. The system of Example 1, wherein the processing circuitry is configured to adjust one or more parameters that define the delivered electrical stimulation based on the received biopotential, wherein the received biopotential is indicative of the patency of the electrode located within the lumen of the anatomical structure.

Example 10. The system of Example 1, wherein the processing circuitry is configured to adjust one or more parameters that define the delivered electrical stimulation based on the received biopotential, wherein the received biopotential is indicative of one or more of: the electrode is operational, the electrode is in a known location, and the electrode is in contact with an anatomical target of interest of the patient.

Example 11. The system of Example 1, wherein the anatomical structure is a blood vessel located in the brain.

Example 12. The system of Example 1, wherein the electrical stimulation circuitry is configured to deliver the electrical stimulation to the patient via the electrode configured to be located within the lumen of the anatomical structure.

Example 13. A method comprising: delivering electrical stimulation to target tissue of a patient; receiving, by sensing circuitry of a medical device, bioelectrical signals from the patient via one or more electrodes proximal to the target tissue, wherein at least one electrode is configured to be located within a lumen of an anatomical structure and proximal to the target tissue; receiving, by processing circuitry of the medical device, and via the sensing circuitry an indication of a biopotential that was elicited by the delivered electrical stimulation, wherein the sensed biopotential is a measurable response indicative of at least one of: a thought pattern from the patient; or a neural state of the patient at the time the electrical stimulation was delivered.

Example 14. The method of Example 13, wherein the medical device is an implantable medical device.

Example 15. The method of Example 13, wherein the medical device comprises the processing circuitry, the method further comprising, controlling, by the processing circuitry, electrical stimulation circuitry of the medical device to deliver the electrical stimulation to the patient.

Example 16. The method of Example 15, further comprising controlling, by the processing circuitry, the electrical stimulation circuitry to deliver the electrical stimulation to the patient via the electrode configured to be located within the lumen of the anatomical structure.

Example 17. The method of Example 15, further comprising controlling, by the processing circuitry, the electrical stimulation circuitry to deliver the electrical stimulation to the patient via a plurality of electrodes configured to be separate from the electrode located within the lumen of the anatomical structure.

Example 18. The method of Example 13, wherein the sensed biopotential is evoked resonant neural activity (ERNA) from the patient.

Example 19. The method of Example 18, wherein the elicited ERNA from the patient exhibits one or more characteristics, and wherein the one or more characteristics comprise a resonant frequency of measured peaks and troughs of the ERNA.

Example 20. A medical device comprising: electrical stimulation circuitry configured to deliver electrical stimulation to a patient via one or more electrodes, wherein at least one electrode is configured to be located within a lumen of an anatomical structure and proximal to target tissue of the patient; sensing circuitry configured to detect bioelectrical signals from the patient via the electrode configured to be located intra-luminally; and processing circuitry configured to: control the electrical stimulation circuitry to deliver electrical stimulation to the patient; receive via the sensing circuitry an indication of a biopotential that was elicited by the delivered electrical stimulation, wherein the sensed biopotential is a measurable response indicative at least one of: a thought pattern from the patient; or a neural state of the patient at the time the electrical stimulation was delivered.

What is claimed is:

1. A system comprising:
an electrode configured to be located within a lumen of an anatomical structure and proximal to target tissue of a patient;
a medical device comprising:
electrical stimulation circuitry configured to deliver electrical stimulation to the patient;
sensing circuitry configured to detect bioelectrical signals from the patient via the electrode; and
processing circuitry configured to:
control the electrical stimulation circuitry to deliver electrical stimulation to the patient;
receive, via the sensing circuitry, an indication of a biopotential that was elicited by the delivered electrical stimulation, wherein the sensed biopotential is a measurable response indicative of at least one of:
a thought pattern from the patient; or
a neural state of the patient at the time the electrical stimulation was delivered.

2. The system of claim 1, comprising a plurality of electrodes separate from the electrode configured to be located within the lumen of the anatomical structure, wherein the electrical stimulation circuitry is configured to deliver the electrical stimulation therapy via one or more of the plurality of electrodes.

3. The system of claim 1, wherein the delivered electrical stimulation elicits a biopotential comprising an evoked resonant neural activity (ERNA) from the patient.

4. The system of claim 3, wherein the target tissue of the patient comprises a location within the patient's subthalamic nucleus (STN).

5. The system of claim 3,
wherein a parameter that at least partially defines the electrical stimulation comprises a stimulation frequency,
wherein the elicited ERNA from the patient exhibits one or more characteristics,
wherein the one or more characteristics comprise a resonant frequency of measured peaks and troughs of the ERNA.

6. The system of claim 1, wherein the delivered electrical stimulation elicits a biopotential comprising an evoked compound action potential (ECAP) from the patient.

7. The system of claim 1, wherein the delivered electrical stimulation is configured to provide stimulation priming signals to target tissue of the patient that enhance the detectability of at least one of:
the thought pattern from the patient; or
the neural state of the patient at the time the electrical stimulation was delivered.

8. The system of claim 1, wherein the processing circuitry is configured to adjust one or more parameters that define the delivered electrical stimulation based on the received biopotential, wherein the received biopotential is indicative of a location of target tissue of the patient for sensing biopotentials that are indicative of thought patterns or neural states.

9. The system of claim 1, wherein the processing circuitry is configured to adjust one or more parameters that define the delivered electrical stimulation based on the received biopotential, wherein the received biopotential is indicative of the patency of the electrode located within the lumen of the anatomical structure.

10. The system of claim 1, wherein the processing circuitry is configured to adjust one or more parameters that define the delivered electrical stimulation based on the received biopotential, wherein the received biopotential is indicative of one or more of:
the electrode is operational,
the electrode is in a known location, and
the electrode is in contact with an anatomical target of interest of the patient.

11. The system of claim 1, wherein the anatomical structure is a blood vessel located in the brain.

12. The system of claim 1, wherein the electrical stimulation circuitry is configured to deliver the electrical stimulation to the patient via the electrode configured to be located within the lumen of the anatomical structure.

13. A method comprising:
delivering electrical stimulation to target tissue of a patient;
receiving, by sensing circuitry of a medical device, bioelectrical signals from the patient via one or more electrodes proximal to the target tissue, wherein at least one electrode is configured to be located within a lumen of an anatomical structure and proximal to the target tissue;
receiving, by processing circuitry of the medical device, and via the sensing circuitry an indication of a biopotential that was elicited by the delivered electrical stimulation, wherein the sensed biopotential is a measurable response indicative of at least one of:
a thought pattern from the patient; or
a neural state of the patient at the time the electrical stimulation was delivered.

14. The method of claim 13, wherein the medical device is an implantable medical device.

15. The method of claim 13, wherein the medical device comprises the processing circuitry, the method further comprising, controlling, by the processing circuitry, electrical stimulation circuitry of the medical device to deliver the electrical stimulation to the patient.

16. The method of claim 15, further comprising controlling, by the processing circuitry, the electrical stimulation circuitry to deliver the electrical stimulation to the patient via the electrode configured to be located within the lumen of the anatomical structure.

17. The method of claim 15, further comprising controlling, by the processing circuitry, the electrical stimulation circuitry to deliver the electrical stimulation to the patient via a plurality of electrodes configured to be separate from the electrode located within the lumen of the anatomical structure.

18. The method of claim 13, wherein the sensed biopotential is evoked resonant neural activity (ERNA) from the patient.

19. The method of claim 18,
wherein the elicited ERNA from the patient exhibits one or more characteristics, and
wherein the one or more characteristics comprise a resonant frequency of measured peaks and troughs of the ERNA.

20. A medical device comprising:
electrical stimulation circuitry configured to deliver electrical stimulation to a patient via one or more electrodes, wherein at least one electrode is configured to be located within a lumen of an anatomical structure and proximal to target tissue of the patient;
sensing circuitry configured to detect bioelectrical signals from the patient via the electrode configured to be located intra-luminally; and
processing circuitry configured to:
control the electrical stimulation circuitry to deliver electrical stimulation to the patient;
receive via the sensing circuitry an indication of a biopotential that was elicited by the delivered electrical stimulation, wherein the sensed biopotential is a measurable response indicative at least one of:
a thought pattern from the patient; or
a neural state of the patient at the time the electrical stimulation was delivered.
